(19) United States
(12) Patent Application Publication
(10) Pub. No.: US 2019/0075756 A1
(43) Pub. Date: Mar. 14, 2019

(54) SYSTEMS, METHODS, AND APPARATUSES FOR ANIMAL WEIGHT MONITORING AND MANAGEMENT

(71) Applicant: FarmIn Technologies, Buenos Aires (AR)

(72) Inventors: Ignacio Luis Albornoz, Mountain View, CA (US); Martin Fabio Garbulsky, Buenos Aires (AR); Diego Manuel Sor, Buenos Aires (AR)

(21) Appl. No.: 16/128,158

(22) Filed: Sep. 11, 2018

Related U.S. Application Data
(60) Provisional application No. 62/556,985, filed on Sep. 11, 2017.

(51) Int. Cl. A01K 11/00; A01K 29/00

(52) U.S. Cl. CPC A01K 11/006; A23L 33/30; A01K 29/00

(57) ABSTRACT

Systems, methods, or apparatuses for animal weight monitoring/managing may comprise at least an identification tag detector, weight sensors, a platform, a localized computing device/remote server, an animal profile database, an analysis module, and/or a graphical user interface. In some examples, the systems, methods, and/or apparatuses may determine an identifier from an identification tag and associate, based on analysis from the analysis module, the identifier with a weight value determined from a weight signal received from the one or more weight sensors. The systems, methods, or apparatuses may improve weight monitoring and management of animals (e.g., bovine cattle) by providing interactive elements for monitoring the weight(s) of one or more animals associated with one or more identifiers, filtering the one more animals into groups for supplier, competitor, and animal health analysis, and presents the results in a manner that improves comprehension of the data and enables scheduling follow-up processes.

### CROSS REFERENCE TO RELATED APPLICATION

This application is based on a prior provisional application Ser. No. 62/556,985, filed on Sep. 11, 2017, the benefit of the filing date of which is hereby claimed under 35 U.S.C. § 119(e), and the entirety of which is hereby incorporated by reference.

### BACKGROUND

Across the world, cattle weighing is highly inefficient and expensive. Standard practice typically involves cattlemen corralling the entire herd into a dedicated weighing pen that houses an expensive and complex alley scale. This process uses a set of procedures and practices that involve forcing the cattle herd to move into unfamiliar and uncomfortable spaces, and also involves the use of certain heavy equipment like passages, clamps, and several other forcing mechanisms. It requires a human workforce to lead these weighing procedures.

The whole operation creates unhealthy stress on the cattle and cowboys alike because everything is done manually—from the passage of cattle through the alley where the scale is settled, to the process for identifying each head of cattle as it is weighed. The typical weighing process faces serious limitations in terms of animal welfare, procedure automation, and the ability to gather useful data regularly, on-demand, or in real-time.

The weighing processes is labor intensive, it provokes disruptions of the herd routine, and it incurs additional time and resource costs. Therefore, cattle operations may typically only weigh cattle three or four times a year, obtaining a very low frequency of data which is difficult to process and turn into useful information. Often, cattle weight data is collected only whenever a certain other “main” procedure is run, and not as an independent activity. With this low level of data collection frequency, in many cases, the data is collected at the herd level, or an average-per-capita weight calculation is used, which is very scarce information to work with for assets—the animals—that can cost around $800-1,300 per head and can constantly fluctuate in weight and health.

The problems faced weighing cattle emerge in a wide variety of animal farming operations such as those involving sheep, goats, horses, llamas, pigs, bovine cattle, and other animals. These problems even arise in wildlife management, zoos, auction houses, or other such operations that have a desire to weigh animals.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items.

FIG. 1 illustrates an example of at least a portion of a system, method, and/or apparatus for monitoring and managing one or more weight(s) associated with one or more animal(s).

FIG. 2 illustrates an example platform and one or more weight sensors, which may form a portion of the system, method, and/or apparatus of FIG. 1.

FIG. 3 illustrates an example identification tag, which may form a portion of the system, method, and/or apparatus of FIG. 1.

FIG. 4 illustrates a schematic diagram of an example system, method, and/or apparatus of FIG. 1, and a localized computing device and/or remote server, which may form a portion of the system, method, and/or apparatus of FIG. 1.

FIG. 5 illustrates a schematic diagram of an example identifier database and an example weight value database, which may form a portion of the system, method, and/or apparatus of FIG. 1.

FIG. 6 illustrates a schematic diagram of an example profile database including an animal profile database and/or a calibration profile database, which may form a portion of the system, method, and/or apparatus of FIG. 1.

FIG. 7 illustrates a schematic diagram of the system of FIG. 1, and an analysis module which may form a portion of the system, method, and/or apparatus of FIG. 1.

FIG. 8 illustrates a schematic diagram of a first example presentation on a graphical user interface and a schematic diagram of a second example presentation on a graphical user interface, which may form a portion of the system, method, and/or apparatus of FIG. 1.

FIG. 9 illustrates a schematic diagram of an example third presentation on graphical user interface, which may form a portion of the system, method, and/or apparatus of FIG. 1.

FIG. 10 illustrates a schematic diagram of an example fourth presentation on a graphical user interface, which may form a portion of the system, method, and/or apparatus of FIG. 1.

### DETAILED DESCRIPTION

This disclosure is directed to systems, methods, and/or apparatuses for improved animal weight monitoring and management. In some examples, a system, method, and/or apparatus may comprise one or more weight sensors fixed to a platform, an identification tag detector for detecting one or more identification tags and determining one or more identifiers associated with the one or more identification tags, a localized computing device and/or a remote server, an animal profile database, an analysis module, and/or a graphical user interface.

In some examples, the system, method, and/or apparatus may detect a presence of an animal (e.g., bovine cattle) standing on the platform via a weight signal from the one or more weight sensors, and/or via an identification signal from the identification tag detector. The system, method, and/or apparatus may receive the weight signal and identification signal at the localized computing device and/or remote server. The system, method, and/or apparatus may determine and store an identifier from the identification signal and a weight value from the weight signal. The system, method, and/or apparatus may associate, via the analysis module, the weight value with the identifier. The system, method, and/or apparatus may present, via the graphical user interface, the weight value, the identifier, the association of the weight value to the identifier, and a variety of other information generated by the analysis module. The system, method, and/or apparatus may provide interactive elements, via the graphical user interface, for viewing information on groups of animals (generated via one or more filters and/or user input), as well as interactive elements for creating alarms, comparing animal statistics received from different suppliers, comparing animal statistics corresponding to different breeds of animals, and scheduling other tasks. Many other features of the system, method, and/or apparatus for animal weight monitoring and management are disclosed herein.

In some examples, the systems, methods, and/or apparatuses disclosed herein may execute herd weight monitoring and management activities, such as weighing one or more animals of the herd in conjunction with a primary herd activity (e.g., drinking water, eating, etc.) in a manner that greatly reduces creating stress situations for the animals of the herd, e.g., by avoiding forcing the animals to go through “special” sites like work pens and passages, sometimes far from the pasture or lot. The systems, methods, and/or apparatuses may collect information about one or more animals of a herd on a regular basis, such as multiple times per day without disturbing the animals' routines. The systems, methods, and/or apparatuses may be used in feedyards and rangeland operations.

In some embodiments, the systems methods, and/or apparatuses may provide weight monitoring of one or more animals on an individual basis. As a result, the systems, methods, and/or apparatuses may monitor and manage entire herd weights, herd sub-group weights, or individual animal weights remotely in real-time or near real-time and provide a reliable source of information as to cattle herd inventory that may be used for, in some examples, commercial sales, auditing, statistical analysis, or regulatory reasons.

In some embodiments, the systems, methods, and/or apparatuses provide information to enable decisions regarding when an animal has completed a growth phase, when an animal has an abnormality in its growth evolution, or when to prepare feeding or other actions to distribute the cattle weight growth in a way that is more cost efficient, logistically simplified, or optimizes a sale price of the cattle (e.g., by showing when the animal has peaked in its growth and value). In some examples, the systems, methods, and/or apparatuses enable a user to oversee the global status of a cattle operation, to monitor compliance of employees regarding their execution of tasks, and provide an accurate portrayal of the location of a herd.

In some embodiments, the systems, methods, and/or apparatuses provide information in a visualized manner such that a user may discover hidden costs or discover and solve nutritional problems during earlier stages when they are more easily solved. In some examples, the systems, methods, and/or apparatuses may build a profile on individual animals of a herd and may schedule or execute a follow up process of one or more animals, or even every animal, of a herd rapidly, such as within the same day that a problem is discovered.

In some examples, the systems, methods, and/or apparatuses may provide a user interface to present information collected or generated by the systems, methods, and/or apparatuses. The user interface may present statistical information on the weight(s) of a herd, and may present the results of analyses that calculate metrics of a whole herd (e.g., the weight distribution of the stock, weight changes over time of the herd, etc.), weight gain performances of different sub-groups (e.g., breeds of cattle, cattle from different suppliers, etc.), or weight change histories of individual animals.

Multiple and varied example implementations and embodiments are described throughout. Some aspects of this disclosure describe systems, methods and apparatuses separately. However, these implementations are merely examples, and portions of the systems, methods, and apparatuses may be rearranged, combined, used together, duplicated, partially omitted, omitted entirely, and/or may be otherwise modified to arrive at variations on the disclosed implementations that combine one or more aspects of the systems, methods, and apparatuses.

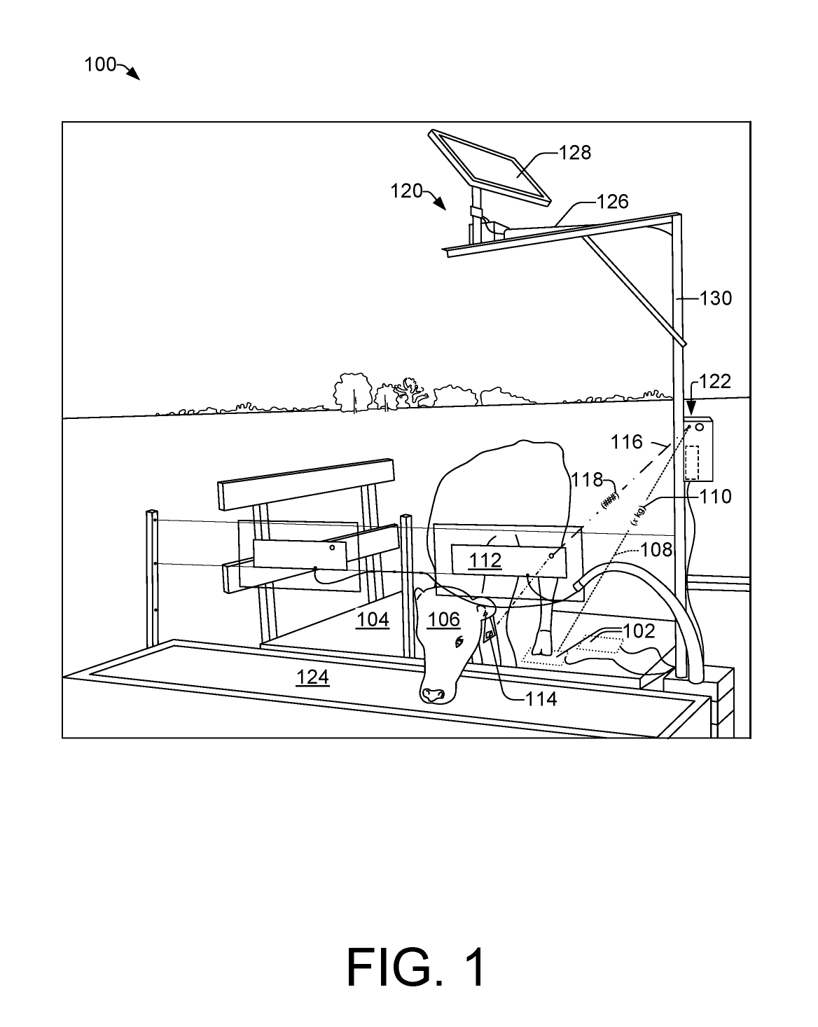

FIG. 1 illustrates an example system, method, and/or apparatus for animal weight monitoring and management (hereafter referred to as “the system” for brevity purposes and not limitation) 100. The system 100 may include one or more weight sensors 102 fixed to a platform 104 for detecting a load, such as one or more animal(s) (hereafter referred to as “the animal,” “the animals,” or “the one or more animals”) 106, which causes the one or more weight sensors 102 to output a weight signal 108 which provides a weight value 110

In some examples, the system may comprise an identification tag detector 112 for detecting and gathering information from one or more identification tags 114 that are associated with one or more animals 106 and outputs an identification signal 116 which provides an identifier 118 that is used to refer to the animal 106 and distinguish it as a particular animal from among a group of animals; a power supply 120 to provide power to one or more components of the system 100

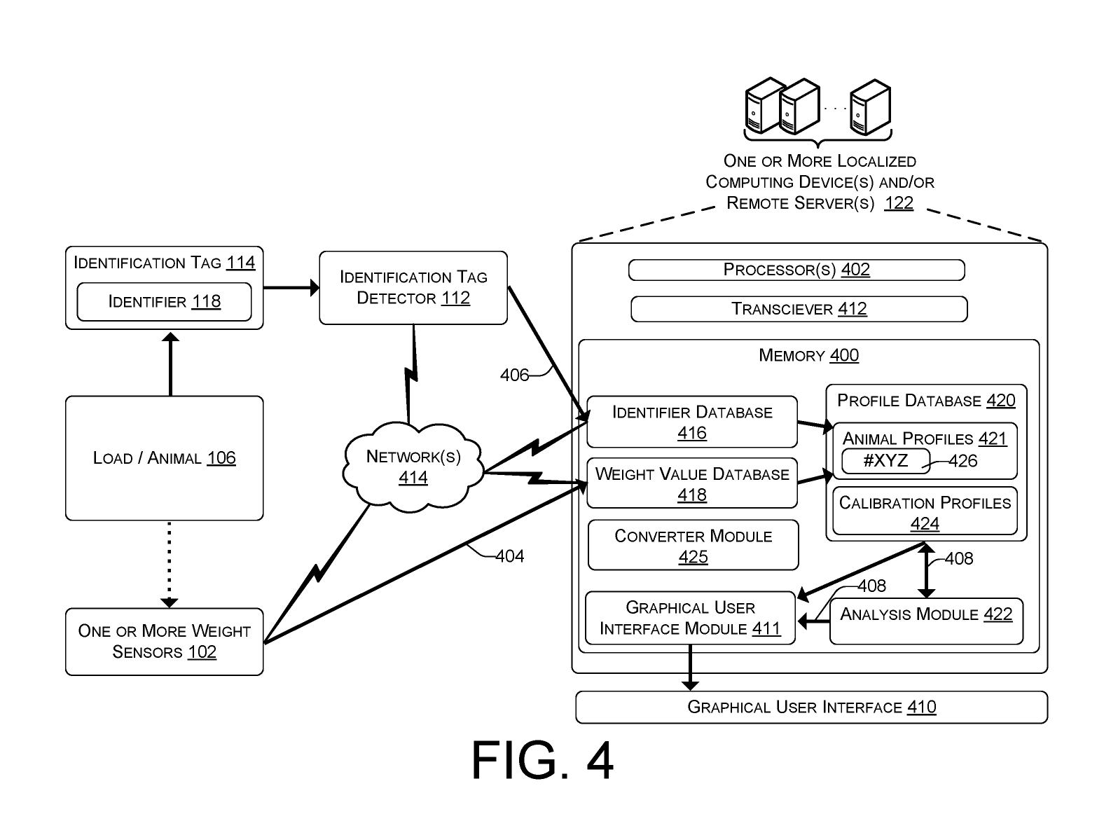

In some embodiments, the system 100 may comprise a localized computer and/or remote server 122 to receive the weight signal 108 from the one or more weight sensors 102 and the identification signal 116 from the identification tag detector 112, to send data between each other, perform analysis on information generated or detected by the system 100, (e.g., the weight value 110, the identifier 118, or other information gathered by sensors of the system 100) and output to a graphical user interface (410 of FIG. 4), output data (408 of FIG. 4) of the analysis, such as an associating the weight value 110 to the animal 106. Each of these components is discussed in greater detail below.

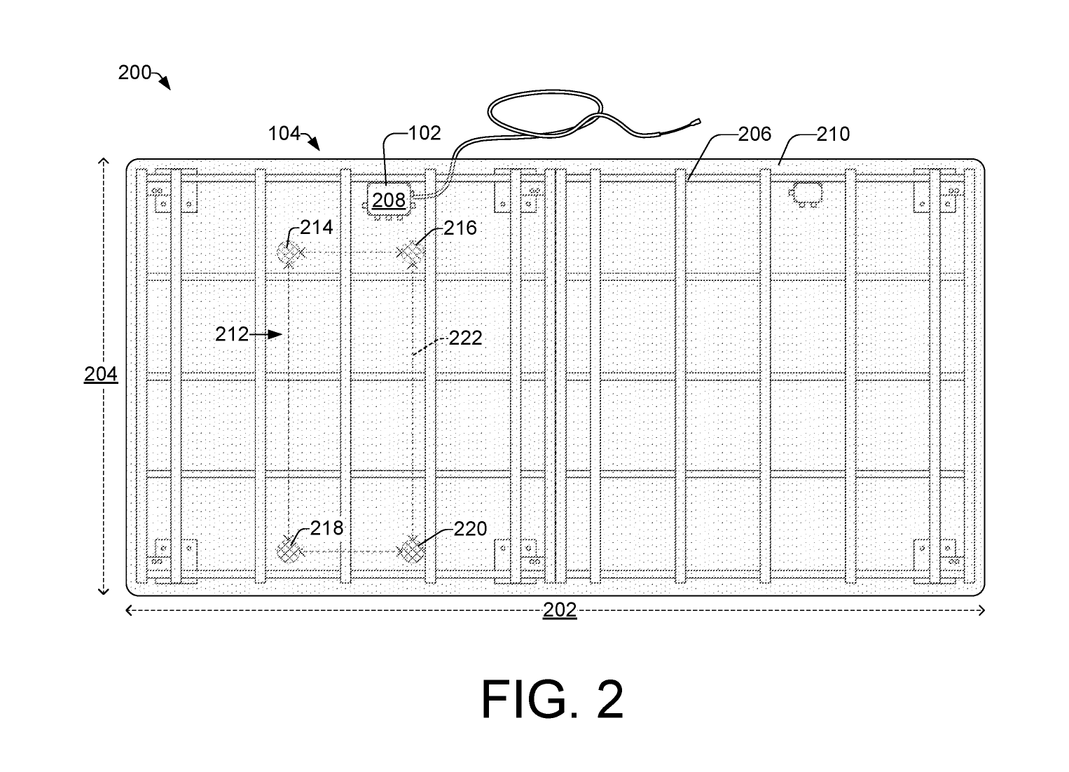

In some examples, the system 100 may comprise the platform 104 to support the one or more weight sensors 106. The platform 104 may be positioned adjacent to a water trough 124, or any other established animal gathering location, such that an animal walks onto the one or more weight sensors 102 when the animal 106 seeks access to the water trough 124. In some instances, the platform 104 with the one or more weight sensors 106 may be placed adjacent to or on top of another established animal gathering location, such as a feed trough, or an area of grazing land (which may be determined to be an established animal gathering location by detecting animal movement patterns over a period of time via GPS trackers, satellite imagery, cameras, other sensors, etc.). In short, an established animal gathering location, like the space adjacent to the water trough 124, is an area animals are naturally likely to congregate during their normal grazing patterns, such that measuring the animal 106 at an area adjacent to or on top of the established animal gathering location avoids disrupting a typical behavior pattern of the animal 106. The platform 104 is described in greater detail below with respect to FIG. 2.

In some examples, the system 100 may comprise the power supply 120. The power supply 120 may provide power to the one or more weight sensors 102, e.g., load cells, as well as to other components of the system 100 (e.g., the identification tag detector 112 and/or the local computing device 122) via one or more electrical wires 126. The power supply 120 may comprise a solar panel 128 attached to a post extending 130 from a base. The base may comprise a compartment for housing other components of the system 100, such as the local computing device 122 or a transceiver (412 of FIG. 4) for sending the weight signal 108 and/or the identification signal 116 to a remote server 122. In some instances the power supply 120 may comprise a battery system, a generator, a wind power system, hydroelectric system, a geothermal generator, or combinations thereof in addition to or alternatively to the solar panel 128. The power supply 120 may comprise a battery system in addition to other sources of power such that the other sources recharge the batteries, and the batteries provide power when the other sources are unavailable (e.g., at night when the solar panel 128 cannot provide direct power). The power supply 120 may comprise an independent power supply such that power is provided to the system 100 in a remote location without access to a municipal electrical grid or other large scale electrical grid systems. In some examples, the power supply 120 provides remote power so that the system 100 operates with minimal or no human involvement.

In some examples, the system 100 may comprise the identification tag detector 112. The identification tag detector 112 may detect a presence of one or more identification tags 114, a distance of the one or more identification tags 114 from the identification tag detector 112, and information stored by the one or more identification tags 114, such as the identifier 118. The identification tag detector 112 may be based on imagery (e.g., a camera system), on radiofrequency technology, or other wireless communication methods (e.g., Wi-Fi, Bluetooth, etc.). In some instances, the identification tag detector 112 may comprise a communication system operating in the ultra high frequency band (UHF) of between 300 megahertz and 3 gigahertz, or at a microwave frequency, e.g., 2.4 gigahertz. In some embodiments, the identification tag detector 112 may comprise an reader electronics board coupled to one or more antennae for detecting the identification tag 114 (for instance, coupled to a body of an animal) positioned above the platform 104. The identification tag detector 112 may be positioned a predetermined distance from the platform 104, that is, the distance necessary for the identification tag detector 112 to be able to detect the identification tag 114 while the identification tag 114 is positioned over the platform 104 and activating the one or more weight sensors 102. The identification tag detector 112 may receive the identification signal 116 from the identification tag 114, and detect, extract, or determine the identifier 118 (e.g., a string of numbers or alphanumeric symbols that indicates a particular animal from a group of animals) from the identification tag 114, or otherwise transform the identification signal 116 into the identifier 118 for storage and/or analysis.

In some examples, the identification tag detector 112 may determine an identity of one or more animals 106 via imagery. For instance, the identification tag detector 112 may implement imagery recognition algorithms to determine an identity of one or more animals 106 positioned on the platform 104. In such scenarios, the system 100 may omit the identification tag 114, and the identity of the one or more animals 106 may be determined directly from the imagery, such that the identification tag detector 112 (which may more aptly be described as an animal identifying/measuring device). For instance, an unmanned aerial vehicle may direct a camera at the animal 106 to determine the identity of the animal 106. In some examples, 3D imagery may determine the identity of the animal 106 and/or the weight of the animal 106 using, in some examples, machine-learning algorithms with a training set of data and/or a neural network computational system to analyze the animal's 106 body dimensions.

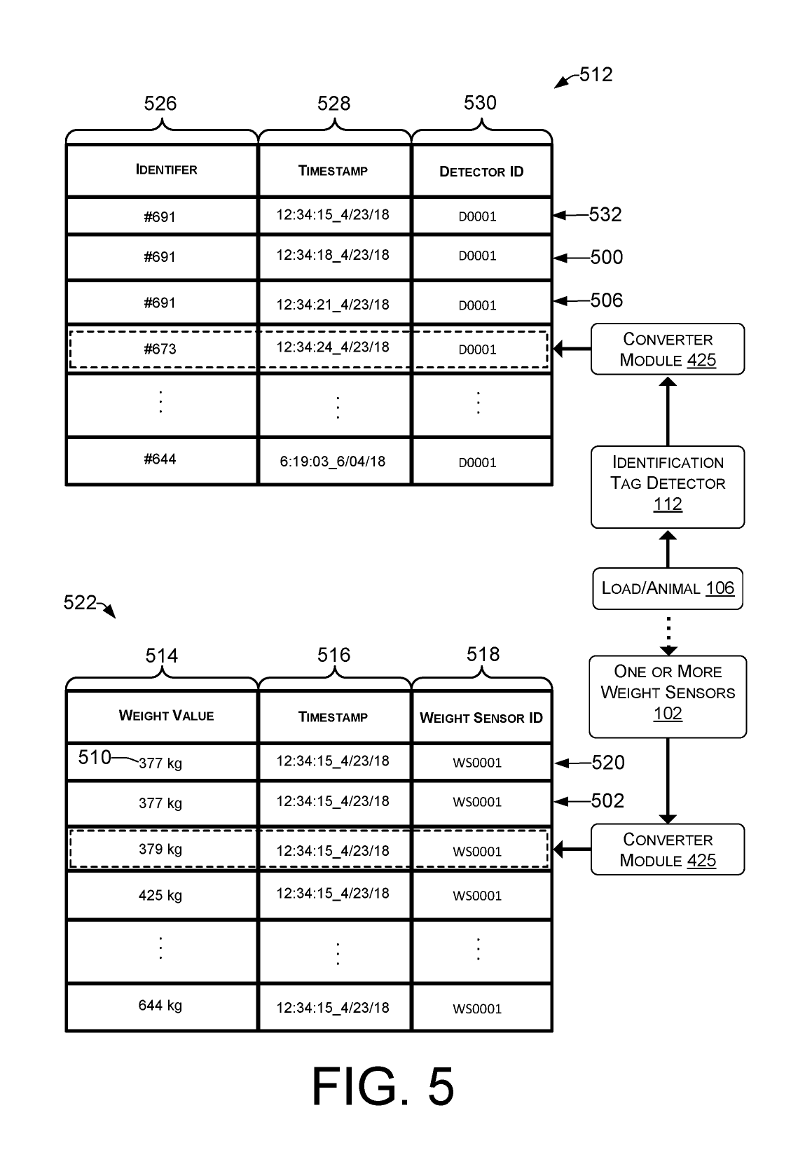

In some examples, the system 100 may comprise a localized computing device and/or a remote server 122 to collect data, analyze the collected data, and output results of the analyses, for instance, to the graphical user interface 410 (illustrated further in FIGS. 4 and 5 below). The localized computing device 122 may comprise a memory (400 of FIG. 4) coupled to a processor (402 of FIG. 4), e.g., via a PCB board or mini-board, and may be situated in proximity to other components of the system 100 (e.g., the one or more weight sensors 102, the identification tag detector 112, and the power supply 120). The localized computing device 122 may be physically wired to the other component noted above such that the localized computing device 122 receives a direct and near instant feed of information from the one or more weight sensors 102 and/or the identification tag detector 112 while receiving power from the power supply 120. The localized computing device 122 may be contained in a weatherproof housing to protect the localized computing device 122 from rain, wind, snow, etc. The localized computing device 122 may perform the receiving, storing, extracting, processing, analyzing, outputting, and/or presenting functions of the system 100, discussed in greater detail below with respect to FIGS. 4-9.

The remote server 122 may comprise a server positioned at a location that is a different location from a location of the other components of the system 100. For instance, the other components of the system 100 may be located at an outdoor site in a pasture with minimal nearby infrastructure, and the remote server 122 may be located in a datacenter, such as a datacenter located in a municipality with access to municipal power and other municipal infrastructure. In some instances, the remote server 122 may be located multiple miles away from the other components of the system 100 (even in a different part of the world), and may receive information wirelessly from a transmitter of the other components (e.g., via a subGhz long-range wireless network, a 915 Mhz signal, Wi-Fi, Bluetooth, 3G, 4G, and/or 5G). The remote server 122 may comprise one or more processors 402, one or more memories 400, and/or one or more virtual machines to perform the processing/analysis functions of the system 100, which are discussed in greater detail below with respect to FIGS. 4-9.

Although the functions of the localized computing device and/or the remote server 122 may be discussed below as primarily occurring at one of the localized computing device 122 or the remote server 122 individually, any function performed by the localized computing device 122 may additionally or alternatively be performed by the remote server 122 and vice versa. Different embodiments may employ different configurations of how data collection, extraction, transforming, processing/analyzing, outputting, and presenting may be allocated between the localized computing device 122 and the remote server 122. Computing resource allocation configurations may be based on respective costs of purchasing, building, and installing localized computing devices 122 versus purchasing remote server 122 access, processing capabilities of localized computing devices 122 versus processing capabilities of remotes servers 122, storage capabilities of localized computing devices 122 versus storage capabilities of remote servers 122, maintenance requirements of localized computing devices 122 versus maintenance requirements of remote servers 122, and/or reliability of localized computing devices 122 versus reliability of remote servers 122. The discussion of FIGS. 4-9 below describes the features and functions of the localized computing device 122 (which also applies to the remote server 122) in greater detail.

In some embodiments, the system 100 may comprise the one or more weight sensors 102 to detect a weight of a load such as the animal 106, or a group of animals, positioned above or standing on the platform 104. The one or more weight sensors 102 may comprise one or more load cells that convert a downward force (e.g., a force in the direction of the ground) into the weight signal 108, e.g., an analog voltage signal, which is, in turn transformed or converted into a weight value 110, which may be digital and stored in one or more memory devices. The one or more weight sensors 102 are discussed in greater detail below with respect to FIG. 2.

In some embodiments, the system 100 may comprise one or more railings extending upward from the platform 104, e.g., from a side of the platform 104, which may guide the animal 106 to the water trough 124, or otherwise constrain the animal onto the platform 104. In some instances, the platform 104, the one or more weight sensors 102, the identification tag detector 112, the power supply 120, the localized computer 122, the one or more railings, and/or one or more mud barriers may be implemented as an integrated structure that may be self-contained. For instance, the system may comprise a frame forming the platform 104 and the one or more rails, and an access opening for providing access to the water trough 124. The one or more weight sensors 102, the identification tag detector 112, the power supply 120, the localized computer 122 may be fixed to and/or housed in the frame. The frame may comprise a single unit, for instance, of tubular metal, metal bars, metal sheets, and/or I-beams welded together, or another rigid structure for supporting the components of the system 100, such that the system 100 comprises a single, integrated, and/or portable unit. In some instances the frame may comprise sliding channels on the platform 104 such that one or more weight sensors 102 (which may be attached to a weight sensor sheet) may be slid into or out of the frame from a sliding opening which, in some examples, may be positioned opposite the access opening. In some instances, the frame may be modular, such that multiple frames may be coupled together and/or detached in order to expand a size of the system 100 or decrease the size of the system. For instance, a side of the frame may comprise one or more coupling mechanisms for attaching the frame to one or more other frames of one or more other systems 100 in, for instance, a side-by-side configuration.

FIG. 2 further illustrates an example weighing system 200 that may form a part of the system 100. In some embodiments, the weighing system 200 may comprise the platform 104 constructed of a rigid material such as a metal, a plastic, a carbon fiber material, a wood, a ceramic material, a composite material, or combinations thereof. The platform 104 may comprise a material and design with a compressive and shear strength to support the one or more weight sensors 102 fixed to the platform 104, as well as multiple full sized animals, e.g., bovine cattle, standing on the weight sensors. In some instances, the platform 104 may support the weight of up to five cows standing on the one or more weight sensors 102 at a single time. The platform 104 may comprise a variety of dimension. For instance, the platform 104 may have a length 202 of six feet or a length 202 of eight feet. The platform 104 may have a length 202 that is a same length as the water trough 124 to which the platform 104 is adjacently positioned. The platform 104 may have a width 204 to accommodate at least four legs of a full a full sized cow, such as a width 204 of eight feet. For instance, the platform 104 may have a width 204 such that the animal 106 must position its entire body on the platform 104 in order to access the water trough 124.

The platform 104 may be substantially planar, in that it extends along a substantially flat plane. In some instances, the platform 104 may comprise a lattice structure 206 of interconnected members. The lattice structure 206 may comprise a triangular lattice, a rectangular lattice (as shown in FIG. 2), or other shaped lattice.

The platform 104 may comprise multiple sub-platforms interlocked together, each sub-platform forming a different plane, as needed to create a platform profile to accommodate uneven variations of the ground surface on which the platform 104 is positioned, such that the platform 104 is firmly secured to the ground surface.

In some embodiments, one or more weight sensors 102 may be fixed to the platform 104. The one or more weight sensors 102 may be fixed below the lattice structure 206 or above the lattice structure 206. The one or more weight sensors 102 may comprise a set of load cells 208 connected via wiring, in some instances, to a PCB board having a mini-controller. In some examples, the one or more weight sensors 102 may be embedded in a ground surface and a cover sheet 210 may be positioned on top of the one or more weight sensors 102 such that the weighing system is at ground level. In some examples, the platform 104 may be placed on the ground surface via mounting feet and a small step-up may provide a path for the animal 106 to step onto the cover sheet when the animal 106 accesses the water trough 124.

In some examples, the animal 106 may step onto the weighing system 200 and activate a particular configuration 212 of the one or more weight sensors 102. For instance, the one or more weight sensors 102 may be arranged as a grid, for instance, with substantially each of the one or more sensors having an associated grid coordinate (e.g., a first sensor 214 may have a grid coordinate (1, 1); a second sensor 216 may have a grid coordinate (1, 2); a third sensor 218 may have a grid coordinate (2, 1); a fourth sensor 220 may have a grid coordinate (2, 2), and so on).

In some embodiments, the animal 106 may activate the particular configuration 212 of the one or more weight sensors 102, such as first sensor 214, second sensor 216, third sensor 218, and fourth sensor 220, when the animal 106 is fully positioned on the platform 104. The one or more weight sensors 102 may output the weight signal 108 caused by the weight of the animal 106 activating the one or more sensors. In some instances, the weight signal 108 may comprise multiple individual signals from individual weight sensors of the one or more weight sensors 102. The one or more of the individual signals may correspond to one or more individual weight sensors, each having a corresponding grid coordinate. Accordingly, the weight signal 108 may comprise information about the particular configuration of the animal 106, generated by multiple weight sensors, and an analysis module (422 of FIG. 4) may process the particular configuration 212 information to determine if it corresponds to predetermined stance dimensions (which may be stored at the localized computing device and/or remote server 122) of the animal 106, e.g., one or more predetermined distances between legs of the animal 106. As such, the system 100 may determine from the weight signal 108 if the animal 106 is fully positioned on the platform 104, if the animal 106 is partially positioned on the platform 104 (e.g., with only one leg, two legs, or three legs on the platform 104), or if multiple animals, fully or partially, are positioned on the platform 104. These functions of the analysis module 422 are discussed in greater detail below with respect to FIGS. 4-9.

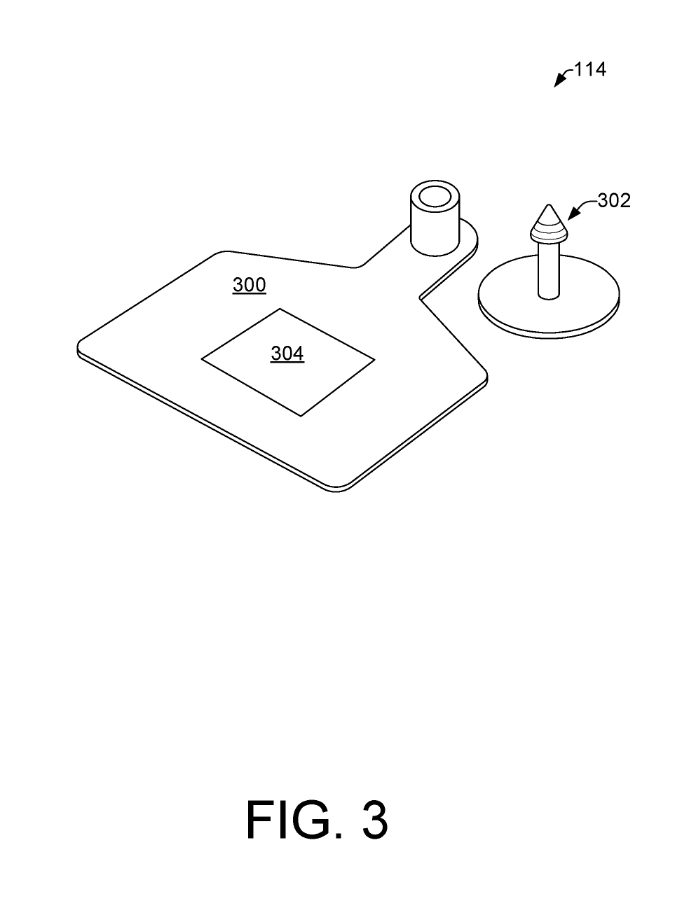

FIG. 3 illustrates an example of the identification tag 114. In some examples, the identification tag 114 may comprise a tag body 300 and a coupling mechanism 302 for attaching the identification tag 114 to a body of the animal 106, e.g., to an ear of the animal 106. The tag body 300 may comprise a readable-information storage component that, when read by a corresponding detector, such as the identification tag detector 112, provides the identifier 118. The identifier 118 may be a digital or analog signal comprising a string of numbers or alphanumeric symbols that are unique to that particular identification tag 114, and the corresponding animal 106 to which the identification tag 114 is attached, at least with respect to other identification tags attached to other animals of the herd. In some instances the identifier 118 may comprise a number, or it may comprise a name of the animal 106. The readable-information storage component of the identification tag 114 may comprise a passive radio frequency identification (RFID) tag 304. In some examples, the readable-information storage component may comprise magnetically stored information and/or a visual indicator.

In some examples, the identification tag 114 may comprise an active RFID tag, such as a battery-powered RFID. The identification tag 114 may comprise a transponder that sends the identification signal 116 in response to receiving a request, or the identification tag 114 may comprise a beacon that sends the identification signal 116 continuously, such as every 3-5 seconds.

In some embodiments, the identification tag 114 may comprise the coupling mechanism 302, such as a rod and hole configuration (e.g., such as an earring) or a magnetic coupling mechanism. In some instances, the identification tag 114 may comprise an injectable readable-chip insertable into the body of the animal 106.

FIG. 4 illustrates an example of the localized computing device 122. The localized computing device 122 may comprise one or more memory devices (“the memory 400”) coupled to a processor 402, e.g., via a PCB board or mini-board. In some implementations, the processor 402 can include a microprocessor, a microcomputer, a microcontroller, a digital signal processor, a central processing unit (CPU), a graphics processing unit (GPU), etc. Among other capabilities, the one or more processors can be configured to fetch and execute computer-readable instructions stored in the memory 400.

In some embodiments, the memory 400 can be non-transitory computer-readable media including, but not limited to, phase change memory (PCM), static random-access memory (SRAM), dynamic random-access memory (DRAM), other types of random access memory (RAM), read only memory (ROM), electrically erasable programmable ROM (EEPROM), flash memory or other memory technology, compact disk ROM (CD-ROM), digital versatile disks (DVD) or other optical storage, magnetic cassettes, magnetic tape, magnetic disk storage or other magnetic storage devices, or any other medium that can be used to store information for access by an electronic device.

The localized computing device 122 may receive information as a first data signal 404 from the one or more weight sensors 102 and a second data signal 406 the identification tag detector 112, analyze the received data signals 404 and 406 to transform them into output data 408, and output the output data 408, for instance, to the graphical user interface 410, for instance, via a graphical user interface module 411. As noted above, some or all of the features discussed above may be performed additionally or alternatively by the remote server 122. For instance, the identification tag detector 112 and/or the one or more weight sensors 102 may include one or more transmitters and receivers (“transceiver 412”) to wirelessly transmit the identification signal 116 and/or weight signal 108 data directly to the remote server 122 via a network 414 (e.g., a subGhz long-range wireless network, a 915 Mhz signal, Wi-Fi, Bluetooth, 3G, 4G, and/or 5G), bypassing the localized computing device 122. Nevertheless, for brevity purposes and not as means of limitation, the features will be discussed primarily with respect to the localized computing device 122.

FIG. 5 illustrates an example portion of the system 100 including receiving the identification signal 116 and transforming the identification signal 116 into an identifier data file 500 stored in the memory 400, and receiving the weight signal 108 and transforming the weight signal 108 into a weight value data file 502 stored in the memory 400. FIGS. 4 and 5 are discussed together in greater detail below.

In some embodiments, the localized computing device 122 comprises one or more processors 402, one or more transceivers 412 (e.g., a transmitter, a receiver, and/or combinations thereof, such as a subGhz long-range wireless network device, a 915 Mhz transmitter, or a Wi-Fi, Bluetooth, 3G, 4G, and/or 5G transmitter), and one or more memory storage devices such as memory 400. The one or more processors 402 may execute one or more modules and/or processes to cause the system 100 to perform a variety of functions, discussed throughout this disclosure. Although some algorithmic functions may be discussed as being performed by a single module, the functions discussed with respect to modules can be performed by separate modules, even independently.

In some examples, the memory 400 may store an identifier database 416, a weight value database 418, a profile database 420, an analysis module 422, and/or a graphical user interface module 411. Furthermore, the profile database 420 may comprise an animal profile database 421 and a calibration database 424.

In some embodiments, the identifier database 416 may store one or more identifiers 118 that are determined from the identification signal 116 received from the identification tag detector 112. In some instances, the identifier 118 may be determined from the identification signal 116 at the identification tag detector 112, and the identifier database 416 may receive the identifier 118. In some instances, the identifier 118 may be determined from the identification signal 116 at the local computing device 122, e.g., by transforming an analog identification signal into a digital identifier data file 500 via a converter module 425. The profile database 420 may comprise an animal profile database 421 which may receive the identifier 118 from the identifier database 416, and may associate the identifier 118 with a previously-stored identifier 506. The previously-stored identifier 506 may be stored in the animal profile database 421 and may be associated with an animal profile 426, so that when the identifier 118 is matched with the previously-stored identifier 506, it is determined that the identifier 118 is stored in the corresponding animal profile 426. In some instances, the localized computing device 122 may omit the identifier database 416, and the animal profile 426 may receive the identifier 118 directly from the identification tag detector 112 or from the converter module 425.

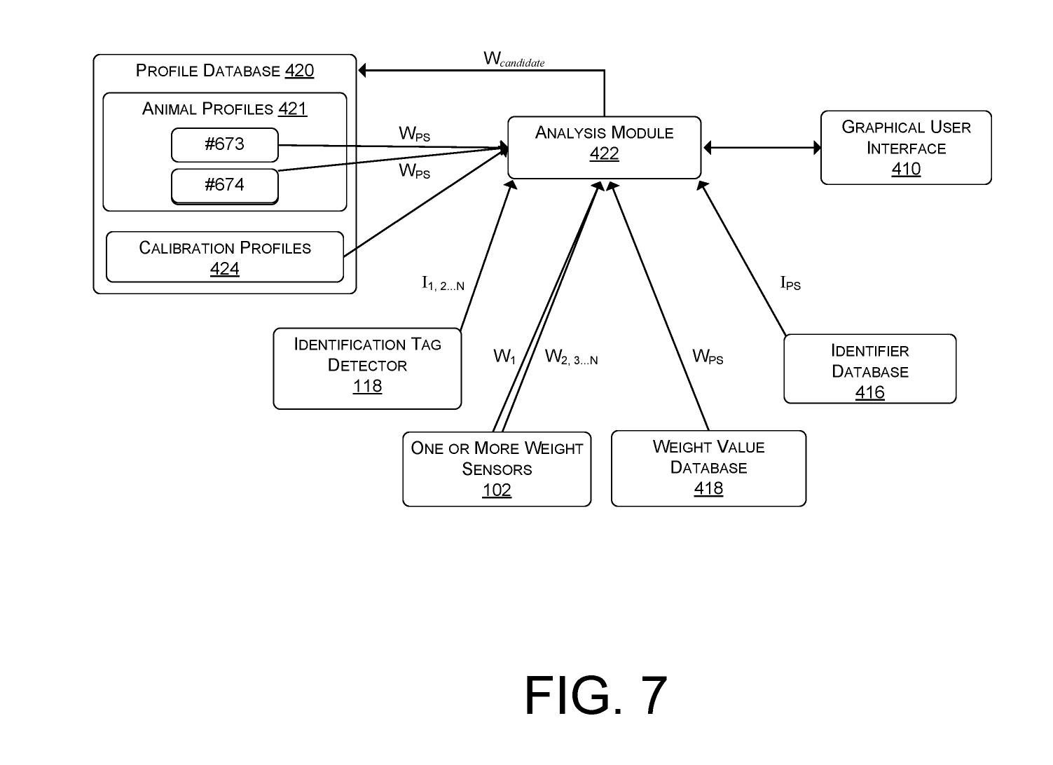

In some embodiments, the weight value database 418 may store one or more weight values 110 that are determined from the weight signal 108 received from the one or more weight sensors 102. In some instances, the weight value 110 may be determined from the weight signal 108 at the local computing device, e.g., by transforming an analog weight signal (e.g., voltage signal) into a numerical weight value 510 via the converter module 425. In some instances, the weight value 110 may be determined from the weight signal 108 upstream from the localized computing device 122, such as at a dedicated converter installed at the one or more weight sensors 102 (e.g., an EEPROM coupled to a processor on a PCB). The analysis module 422 may perform analysis on the weight value 110 and determine an association between the weight value 110 and the identifier 118, e.g., by determining that a timestamp associated with the identifier 118 is within a predetermined time range of a timestamp associated with the weight value 110, and/or by determining that a location ID of the identifier 118 corresponds to a location ID of the weight value 110. Analysis techniques performed by the analysis module 422 to associate the weight value 110 with the identifier 118 are discussed in greater detail below with respect to FIG. 7.

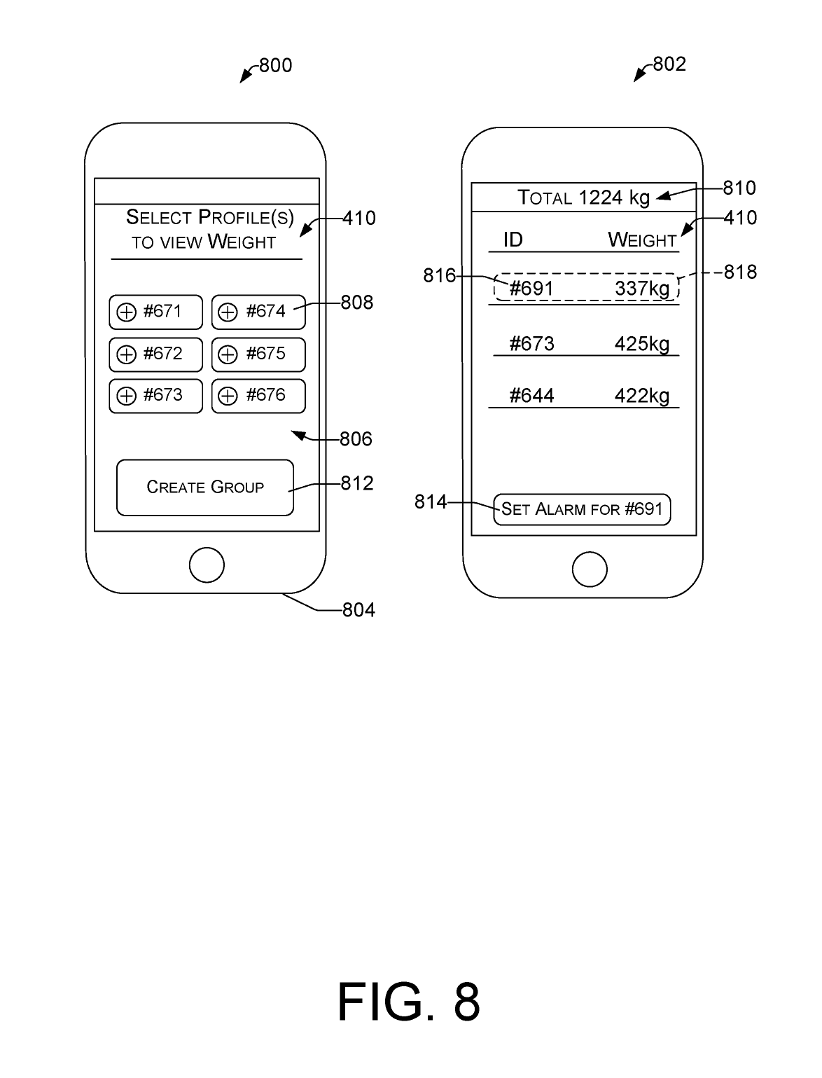

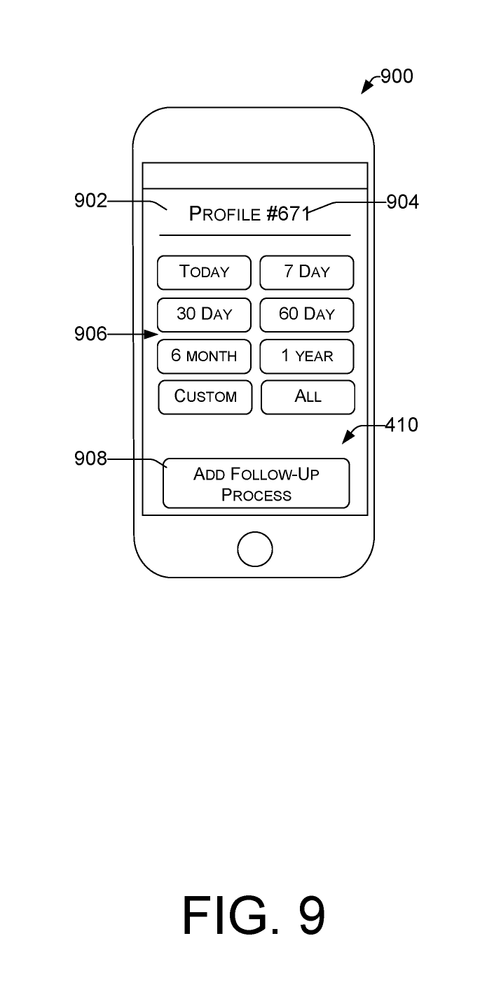

In some embodiments, upon associating the weight value 110 with the identifier 118, the weight value 110 may be stored in an animal profile 426 as associated with the identifier 118. The localized computing device 122 may receive a request to present the weight value 110, as associated with an animal corresponding to the identifier 118, on the graphical user interface 410. The request, itself, may be received from the graphical user interface 410, or the request may be received from a second, different graphical user interface 410, or the request may be received from an input unrelated to the graphical user interface 410, such as voice command via a phone call, or a physical gesture detected by a motion sensor. Presentation of output data 408 created by the analysis module 422 is discussed in greater detail below with respect to FIGS. 8 and 9. Functions described with respect to the localized computing device and/or remote server 122 may be fragmented and performed individually or separately at device-specific computers associated with one or more of the one or more weight sensors 102, the weighing system 200, the identification tag detector 112, the identification tag 114, the power supply 120, or combinations thereof.

In some embodiments, the weight value database 418 may comprise one or more spreadsheets 512 and/or other data structures that store one or more weight value data files 502 comprising one or more weight values 110 and information associated with the one or more weight values 110. The spreadsheet 512 may comprise row corresponding to each of the one or more weight value data files 502, and a column to correspond to an association of information of the weight value data file 502. For instance, the spreadsheet 512 may comprise a first column 514 comprising a weight value column, a second column 516 comprising a timestamp column, and a third column 518 comprising a weight sensor ID column. Additional columns may be included to store other information associated with the weight value 110 (e.g., a duration of weight measurement, an error range of weight measurement, GPS coordinates, user-entered information).

In some embodiments, each row of the weight value database 418 may store the weight value 110 and information associated with the weight value 110 as the weight value data file 502. For instance, a first row 520 may store a first weight data file including, by way of example, the particular weight value “377 kg” in the first column 514. The first row 520 may store, by way of example, a particular timestamp “12:34:16_4/23/18” in the second column 516, indicating a time and date that the weight value 110 is received by the weight value database 418, or indicating a time that the weight value 110 or the weight signal 108 from which the weight value 110 is generated. The first row may store 520, by way of example, a particular weight sensor ID “WS0001” indicating an identity of one or more weight sensors 102 from which the weight value 110 originated. In some instances, the weight sensor ID may correspond to a physical geographical location. In some instances, the analysis module 422 may associate the weight value 110 with the identifier 118 by determining that the weight sensor ID corresponds to the identification tag detector ID, e.g., which may lead to the determination that both the weight sensor ID and the identifier ID correspond to a same geographical location. GPS beacons may also provide location data from the identification tag detector 112 and the weight sensor for this purpose.

In some embodiments, the identifier database 416 may comprise one or more spreadsheets 522 and/or other data structures that store one or more identifier data files 500 comprising one or more identifiers 118 and information associated with the one or more identifiers 118 (as illustrated in FIG. 5). The spreadsheet 522 may comprise a row corresponding to each of the one or more identifier data files 500, a first column 526 comprising an identifier column, a second column 528 comprising a timestamp column, and a third column 530 comprising a detector ID column. Additional columns may be included to store other information associated with the identifier 118 (e.g., GPS coordinates, supplier, breed, purchase date, optimal weight, user-entered information).

In some embodiments, each row of the identifier database 416 may store the identifier 118 and information associated with the identifier 118. For instance, a first row 532 may store, by way of example, the particular identifier “#691” in the first column 526. The first row 532 may store, by way of example, a particular timestamp “12:34:15_4/23/18” in the second column 528, indicating a time and date that the identifier 118 is received by the identifier database 416, or indicating a time and date that the identifier 118 is determined by the identification tag detector 112. The first row 532 may store in the third column 530, by way of example, a particular detector ID “D0001” indicating an identity of the identification tag detector 112 from which the identifier 118 originated. In some instance, the detector ID may correspond to a physical geographical location.

The identifier 118 may be added to the identifier database 416 in addition to one or more previously-stored identifiers 506. In some examples, the analysis module 422 may use the identifier 118 and a collection of previously-stored identifiers 506 in making various determinations or associations, often to generate historical and/or up-to-date, accurate statistics about animal weight, animal health, animal locations, herd patterns, supplier performance, and breed performance, as discussed in greater detail below with respect to FIG. 7.

Although the spreadsheets 512 and 522 are discussed herein, the identifier database 416 and/or the weight value database 418 may, additionally or alternatively, be stored as a comma delimited list, a NoSQL data structure, or any other data type, data structure, and/or data system.

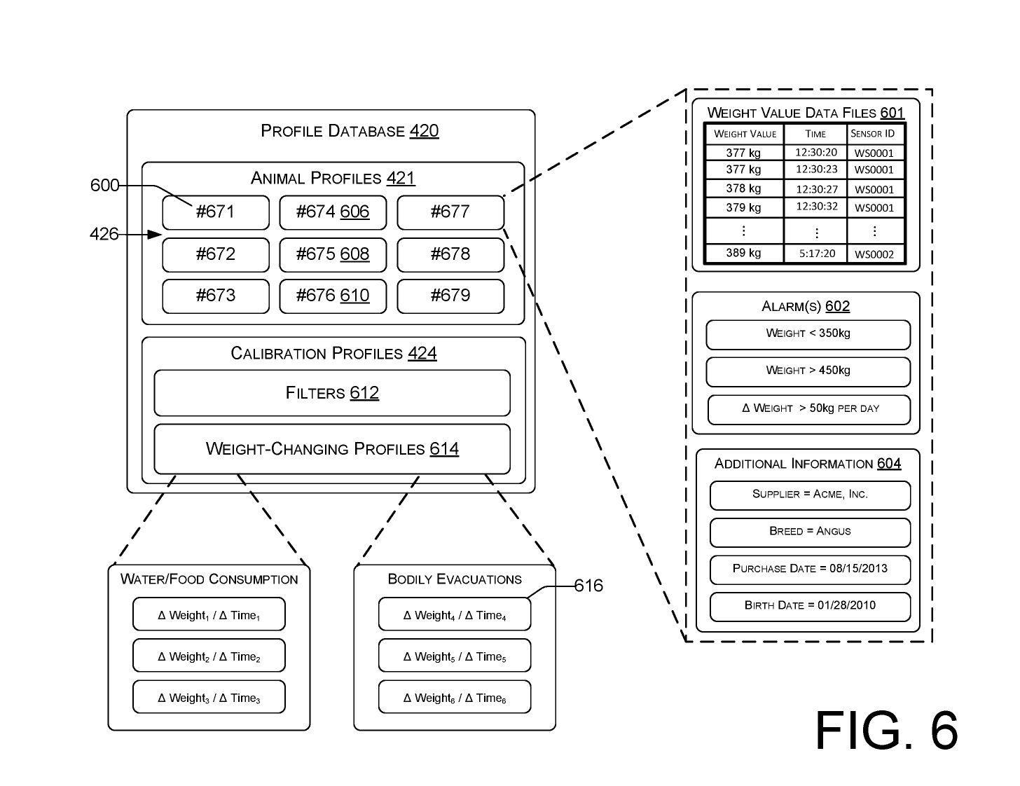

FIG. 6 illustrates an example profile database 420 including an animal profile database 421 and a calibration profile database 424. The animal profile database 421 may store one or more animal profiles 426. The animal profile 426 may comprise a directory or data file storing information associated with a particular animal, collected over a period of time. For instance, the animal profile 426 may include a particular identifier 600 associated with the animal profile 426. Each animal profile 426 may include the particular identifier 600 that is unique to that animal profile 426. In some instances, when the weight value 110, or any other data, is associated with the identifier 118, the identifier 118 may be matched to a particular identifier 600 of the animal profile 426 in order to determine to which animal profile 426 the weight value 110, or any other data, is associated, and subsequently stored, often for future processing and analyses.

In some examples, the animal profile 426 may store a collection of weight value data files 601 associated with the identifier 118 of the animal profile 426. For instance, the animal profile 426 may store one or more weight value data files 502 similar to those stored in weight value database 418 discussed above for each, some, or every weight value 110 that is associated with the identifier 118. The collection of weight value data files 601 may include one or more weight values 110, one or more time stamps associated with the one or more weight values 110, and/or one or more weight sensor IDs associated with the one or more weight values 110. In some examples, much information can be determined from the collection of weight value data files 601, e.g., via the analysis module 422, such as a trend of weight gain or loss corresponding to the particular animal associated with the animal profile 426; or a movement of the particular animal from a first location to a second location by determining that a first weight value corresponds to a first weight sensor ID at a first time, and a second weight value corresponds to a second weight sensor ID that is different than the first weight sensor ID at a second time. The analysis module 422 may detect patterns of weight gain or loss or patterns of movement from the collection of weight value data files 601 associated with the animal profile 426.

In some instances, the associating may comprise storing the weight value 110 in a same animal profile 426 as the identifier 118, adding a column or data entry location in the weight value data file 502 for indicating the association with the identifier 118, adding a column or data entry location in the identifier data file 500 for indicating the association with the weight value 110, or otherwise storing an association indicator in the memory 400 that is retrievable to recall the association when queried regarding the weight value 110, the identifier 118, or other information associated with the weight value 110 or identifier 118.

In some embodiments, the animal profile 426 may store one or more alarms 602 corresponding to the animal profile 426. For instance, the alarm 602 may associated with the particular animal of the animal profile 426 and may be set via the graphical user interface 410, discussed in greater detail below with respect to FIG. 8. In some examples, the alarm 602 may be set to trigger and provide a notification (e.g., a sound alert, a visual alert such as a red bubble or red number alert, a tactile alert such as a vibration, etc.) if a preset parameter or event occurs. For instance, the alarm 602 may be set to trigger if the weight value 110 associated with the particular animal falls below a predetermined value, e.g., 350 kg, or the weight value 110 is greater than a predetermined value, e.g., 450 kg. In some instances, the alarm 602 may be set to trigger if a predetermined weight change occurs within a particular time period, e.g., 50 kg per day.

In some embodiments, the animal profile 426 may include additional information 604 corresponding to the animal 106 associated with the animal profile 426. For instance, the additional information may include a supplier identity of the animal 106, a breed of the animal 106, a purchase date of the animal 106, and/or a birthdate of the animal 106. The additional information 604 may be accessed and processed by the analysis module 422 to generate output data 408 for presentation on the graphical user interface 410, and/or to set alarms 602, as discussed in greater detail below with respect to FIGS. 8-10.

In some examples, the animal profile 426 may comprise information associated with a group or sub-group of animals. For instance a first animal profile 606 associated with a first particular animal may be combined with a second animal profile 608 associated with a second particular animal to create a third animal profile 610 that is an animal group profile associated with the first particular animal and the second particular animal. In some instances, the animal group profile may be associated with a collection of animals based having a same supplier, a same breed, a same purchase date (or range of purchase dates), a same weight value (or range of weight values), or a same location. Information associated with the animal group profile (e.g., average weight values, trends in weight value changes, location(s), or other additional information associated with the animal group profile may) be presented on the graphical user interface 410 in response to a receiving a request, for instance, from an interactive element of the graphical user interface 410.

In some embodiments, the profile database 420 may comprise a calibration database 424. The calibration database 424 may store one or more filters 612 and/or weight-changing profiles 614, which may be used by the analysis module 422 to increase an accuracy of calculations and determinations made by the analysis module 422. For instance, one or more filters 612 may comprise values that are added or subtracted to measured weight values 110 by the analysis module 422 during analysis/calculations to account for a temperature impact on the one or more weight sensors 102, a moisture impact on the one or more weight sensors 102, or any other physical impact that may affect an accuracy of the weight measurements. For instance, if a particular weight sensor detects a continuous reading of 20 kg over a predetermined time, e.g., 24 hours, a filter value may be created to subtract 20 kg from every measurement from that particular weight sensor. The analysis module 422 may determine that the weight signal 108 is a continuous weight signal below a certain weight threshold and may create a filter to account for the weight signal 108, and/or the analysis module 422 may determine that an object (e.g., a tree branch or animal carcass) has come to rest on the weight sensor, causing the continuous weight signal. The analysis module 422 may determine that a continuous weight signal from the one or more weight sensors 102 is generated in the absence of the identification tag 114 and should, therefore, be subtracted from other weight signals generated from the one or more weight sensors 102 in the presence of the identification tag 114. The analysis module 422 may determine, based on the weight signal 108 being generated in the absence of the identification tag 114, that the weight signal 108 corresponds to an animal that is not a target animal (e.g., a wild animal such as a coyote) and, therefore, the analysis module 422 may determine that the weight signal 108 is not associated with the identifier 118.

The system 100 may continuously run arithmetic equations incorporating newly generated data to continuously create new filters and recalibrate the one or more weight sensors 102 in order to maintain an accuracy of the weight signal 108 generated by the one or more weight sensors 102 when the identification tag 114 is present. In other words, the system 100 may provide automatic tare functionality to take into account physical changes occurring at the one or more weight sensors 102, debris resting on the one or more sensors, and/or wild animals activating the one or more sensors.

In some instances, the calibration database 424 may comprise one or more weight-changing profiles 614. The weight-changing profiles 614 may comprise stored data files of a particular weight-change over a particular time that reflects commonly occurring events related to the animal 106. For instance, the weight-changing profile 614 may comprise an increase of 40 kg over 5-20 minutes which may relate to the animal 106 consuming water. The weight-changing profile 614 may comprise a decrease of 20 kg over 1-10 minutes which may relate to the animal 106 having a bodily evacuation (bowel movement and/or urination). The weight-changing profile 614 may account for feed intake, water intake, perspiration, and/or any other animal event that affects the weight of the animal 106. In some instances, the analysis module 422 may detect a weight-changing event at the one or more weight sensors 102, determine that the identification tag 114 is present at the one or more sensors, match the weight changing event with the weight-changing profile 614 stored in the calibration database 424, and as a result, associate the weight changing event with the animal 106 associated with the identifier 118 of the identification tag 114. Accordingly, the weight value 110 may be increased or reduced to account for the weight-changing event, and/or the weight-changing event may be stored in the animal profile 426 associated with the animal 106 as a weight-changing profile 616.

FIG. 7 illustrates a portion of the system 100 that comprises an analysis module 422 for analyzing one or more weight signals 108 and/or one or more identification signals 116 received by the analysis module 422, transforming the received signals into output data 408, and outputting the output data 408 to, in some instances, a graphical user interface 410. In some instances, the analysis module 422 may output the output data 408 to the profile database 420 (e.g., the animal profile database 421 or the calibration profile database 424), such that it may be used in subsequent analyses, e.g., in an iterative process. Many functions of the analysis module 422 are discussed above.

In some examples, the analysis module 422 may receive input data from the identification tag detector 112, the one or more weight sensors 102, the weight value database 418, the identifier database 416, one or more animal profiles 426, and the calibration database 424. The analysis module 422 may perform multiple functions, such as associating the weight value 110 with the identifier 118, improving an accuracy of the weight value 110, and generating output data 408, either automatically or upon receiving a request, to the graphical user interface 410.

In some embodiments, as discussed above, the analysis module 422 may associate the weight value 110 with the identifier 118 in order to correspond the weight value 110 to the animal 106 associated with the identifier 118. For instance, the analysis module 422 may receive a first weight value W₁ from the one or more weight sensors 102 and a first identifier from the identification tag detector 112. A first timestamp may be associated with W₁ and a second timestamp may be associated with I₁, and the analysis module 422 may determine that the first timestamp and the second timestamp are within a predetermined range (e.g., 0.5 seconds 1 second, 2 second, 3, seconds, 5 seconds, or 30 seconds), which may be based on a lag time, refresh rate, processing capability, or device limitation, such that the analysis module 422 determines that the first W₁ and I₁ were generated substantially contemporaneously. Upon determining that W₁ and I₁ were generated substantially contemporaneously, the analysis module 422 may determine that W₁ is associated with I₁.

In some instances, I₁ may include a identification detector ID and/or first a location data, and W₁ may include a sensor ID and/or second location data. The analysis module 422 may determine that the first location is within a predetermined distance of the second location (e.g., 0-10 meters), and associate W₁ with I₁ based on this determination. In some instances, W₁ may be associated with I₁ based on a substantially contemporaneous determination and the location determination.

In some examples, the analysis module 422 may receive W₁ from the one or more weight sensors 102 and one or more previously-stored weight values W_{PS} from the animal profile 426, for instance, from animal profile #673. The analysis module 422 may determine that W₁ is within a predetermined threshold range, (e.g., 10 kg, 20 kg, or 30 kg) of W_{PS} and, based in part on this determination, associate W₁ with the animal profile 426 storing W_{PS}, for instance animal profile #673. In some instances, the analysis module 422 may compare Wi to multiple previously-stored weight value data files W_{PS} from one or more animal profiles 426 to determine a closest previously-stored weight value, and associated the weight value W₁ with the animal profile 426 corresponding to the closest previously-stored weight value.

In some examples, the analysis module 422 may receive the weight value 110 from the one or more weight sensors 102 and may determine that the weight value 110 corresponds to a group of animals. For instance, the analysis module 422 may receive a plurality of identifiers I_{1, 2, . . . N} from the identification tag detector 112 substantially contemporaneously with the weight value W₁, and, based on the fact that more than one identifier 118 corresponds temporally and/or spatially (e.g., based on location data associated with W₁ and location data associated with I_{1, 2, . . . N}) with the weight value W₁, the analysis module 422 may determine that a group of animals is activating the one or more weight sensors 102. In some instances, the analysis module 422 may determine that the group of animals is activating the one or more weight sensors 102 based on the weight value W₁ being greater than a threshold value. For instance, the analysis module 422 may determine that the weight signal 108 includes the weight value W₁ that is greater than a maximum value of a particular type of animal (e.g., greater than 815 kg for bovine cattle) and, based on this determination, determine that the load activating the one or more weight sensors 102 comprises two or more of the animal 106.

In some instances, the analysis module 422 may determine that the group of animals is activating the one or more weight sensors 102 based on a series of increasing weight values 110, each of the weight values 110 being within a range indicative of a particular type of animal (e.g., between 38 kg and 815 kg for bovine cattle). For instance, the analysis module 422 may receive the first weight signal W₁ including a first weight value, e.g., 450 kg, and subsequently receive a second weight signal W₂ including a second weight value, e.g., 925 kg. The analysis module 422 may determine that, based on the received order and weight values 110 of the weight signals W₁ and W₂, that the second weight signal W₂ indicates the presence of two animals: a first animal weighing 450 kg (Wi), and a second animal weighing W₂ minus W₁, i.e., 375 kg. The analysis module 422 may determine that a voltage signal from the one or more weight sensors 102 comprises the weight signal 108 by determining that the voltage signal reaches a substantially equilibrium value, e.g., by not changing outside an error range for a particular time interval, indicating a stabilized weight load on the one or more weight load sensors. After a series of stabilized weigh loads results in a series of weight signals 108, the analysis module 422 may calculate various possible candidate weight values W_{candidate} by subtracting lessor weight signals from greater weight signals (as in the above example) and/or the analysis module 422 may compare one or more received weight values 110, or calculated candidate weight values W_{candidate}, to previously-stored weight values W_{PS} associated with individual animal profiles 426. The analysis module 422 may perform this analysis in an iterative “guess-and-check” manner, adding and subtracting various weight values 110, as well as calibration values, animal event values, and or previously-stored weight values W_{PS}, and find a match between the received weight value W₁ or the calculated candidate weight value W_{candidate}, and a weight range associated with the previously-stored weight value W_{PS}.

In some instances, the above process may include cross-correlating one or more identifiers 118 received in a temporal series with a series of weight values W_{2, 3 . . . N} in order to associate one or more weight values 110 of the series of weight values W_{2, 3 . . . N} with the one or more identifiers 118. For instance, at a first time, W₁ may be received and I₁ may be received. At a second time, W₂ may be received and I₁ and I₂ may be received (indicating that W₂ corresponds to a sum of weight values associated with I₁ and I₂). At a third time, W₃ may be received and I₁, I₂, and I₃ may be received (indicating that W₃ corresponds to a sum of weight values associated with I₁, I₂, and I₃). At fourth time, W₄ may be received and I₂, and I₃ may be received (indicating that an animal associated with I₁ has left the one or more weight sensors 102, and W₄ corresponds to a sum of weight values associated with I₂, and I₃). Various other combinations of identifiers, and changes of combinations of identifiers may be detected and correlated to weight signals 108, such that the analysis module 422 may use arithmetic, while, in some instances, accessing previously-stored weight values W_{PS} and calibration profiles, to associate a plurality of identifiers (indicating a sub-group of animals on the platform 104) with the weight signal 108, and to determine individual weight values associated with individual identifiers of the plurality of identifiers.

In some embodiments, the analysis module 422 may generate output data 408 as a result of analysis performed by the analysis module 422. For instance, output data 408 may comprise a calculation of the candidate weight value W_{candidate} (e.g., extracting an individual weight value from a group-of-animals weight value as discussed above and/or calculating a weight value 110 from the weight signal 108 and calibration data files), an association of a weight value 110 with the identifier 118, or a filtered list (e.g., a list of animals within a weight range, a list of animals from a particular breeder, a list of animals purchased on a particular date, a list of animals having a particular weight change over a particular time period, a list of animals in a particular location at a particular time).

In some examples, W₁ may comprise one or more grid coordinates associated with one or more weight signals 108 received from the one or more weight sensors 102 (as discussed in FIG. 3 above). The analysis module 422 may compare one or more distances 224 between the one or more grid coordinates of the particular configuration 212 of activated weight sensors with a predetermined stance dimensions 222 of the animal 106 to determine if W₁ is associated with the animal 106 fully standing on the one or more weight sensors 102, or partially standing on the one or more weight sensors 102. A number of individual weight signals from the particular configuration 212 of activated weight sensors may indicate a number of legs present on the one or more weight sensors 102. For instance, the analysis module 422 may determine that multiples of four weight signals (e.g., 4, 8, 16, 20, etc) indicate one or more four-legged animals fully standing on the one or more weight sensors 102, whereas a non-multiple of four weight signals, (e.g., 1, 2, 3, 5, etc.) indicate one or more animals partially positioned on the one or more weight sensors 102, e.g., standing with only one leg on the platform 104. The analysis module 422 may determine to conduct analysis on the weight signal 108 based on determining that the animal 106 is fully standing on the one or more weight sensors 102, or the analysis module 422 may determine that the weight signal 108 does not represent a weight of one or more animals fully standing on the platform 104 based on determining that a non-multiple of four legs is present on the platform 104.

In some embodiments, the analysis module 422 may conduct multiple of the analysis techniques disclosed herein, and may base the association of the weight value 110 with the identifier 118 on a combination of the multiple analysis techniques. For instance, the analysis module 422 may assign a coefficient to an analysis technique to give the analysis technique a weighted impact on the combined analysis. In some examples, the analysis module 422 may adjust one or more coefficients assigned to the one or more analysis techniques based on a confirmation that one or more of the analysis techniques provides accurate results relative to others of the one or more analysis techniques. In this way, the analysis module 422 may use an iterative process to continually improve the accuracy of the output data 408. Although the analysis module 422 may be discussed herein as a single module, the analysis module 422 may comprise multiple modules performing multiple analyses at multiple, different locations and/or times, and may operate in communication with each other and/or independently.

FIG. 8 illustrates an example first graphical user interface presentation 800 and an example second graphical user interface presentation 802. The graphical user interface 410 may comprise an interactive or non-interactive visual presentation of data collected by the system 100, output data 408 generated by the analysis module 422, and information entered into the system 100 by a user of a device 804. The presentations may be displayed on the device 804, such as a mobile device, a laptop computer, a desktop computer, a smart wearable device (e.g., a smart watch, smart glasses, a smart bracelet, a smart necklace, an epidermal device) an Internet of Things home device, or any other type of device or electronic device having a visual display for human consumption of and/or interactions with data. In some examples, the presentations may be provided to an interface with non-visual elements, such as a voice command/audio interface, or an interface with a combination of visual and non-visual elements.

In some embodiments, the device 804 for presenting the graphical user interface 410, in combination with other elements of the system 100 discussed herein, represents a particular, non-conventional arrangement of components (e.g., hardware components, software components, and combinations thereof) that operate in a non-conventional matter to solve specific problems in managing and displaying the data collected and generated by the system 100 (e.g., animal weight data and animal identification data), and for generating new data that previously did not exist prior to interactions with the device 804 and/or system 100.

In some examples, the graphical user interface 410 may present one or more interactive elements 806 that provide access to a visual representation of data stored in the one or more animal profiles 426. For instance, interaction with an interactive element 808 may cause the graphical user interface 410 to display the identifier 118 associated with the animal profile 426, and the weight value 110 associated with the animal profile 426, such that the presentation on the graphical user interface 410 comprises displaying the weight value 110 associated with the animal 106. In some examples, the identifier 118 may be converted to a name (e.g., “Sally”) of the animal 106 for presentation with the weight value 110, or the identifier 118 may be presented as a number. In some instances, the weight value 110 may comprise a most recently received weight value associated with the animal profile 426 (as determined by comparing a timestamp associated with the weight value 110 to a current-time indicator clock). The weight value 110 may be presented as a weight in units of kilograms, pounds, ounces, or other weight units. In some examples, the graphical user interface 410 may present an interactive element to change the presented weight units, and the converter module 425 may convert a first weight unit into a second weight unit (e.g., converting kilograms into pounds by multiplying a kilogram value by 2.205).

In some instances, the graphical user interface 410 may present a plurality of interactive elements representing a plurality of animal profiles which, upon interaction, display a plurality of identifiers with a corresponding plurality of weight values (e.g., up-to-date/current weight values) on the graphical user interface 410 together. In some instances, the graphical user interface 410 may present a total weight value 810 representing a sum of the plurality of weight values.

In some examples, the graphical user interface 410 may present an interactive element 812 for, upon interaction with the interactive element 812, creating a group profile. An interaction with the interactive element 812 may display one or more attribute fields and/or toggles that receive user input for determining one or more group filters, which may apply to the one or more animal profiles 426 in order to return a list of identifiers 118 and/or animal profiles 426 that satisfy the one or more group filters. For instance, the one or more group filters may correspond to animals 106 in a current particular weight range (the weight range being generated by user input or by a pre-set range value), animals 106 purchased from a particular or a combination of breeders, animals 106 purchased on a particular date or a combination of dates, animals 106 having a particular weight change over a particular time period, and/or animals 106 in a particular location at a particular time.

In some instances a first group corresponding to a first set of group filters may be created via the graphical user interface 410, and a second group corresponding to a second set of group filters may be created via the graphical user interface 410. Animals 106 of the first group and/or information associated with the first group may be displayed, and animals 106 of the second group and/or information associated with the second group may be displayed, such that the first group is presented with the second group as a side-by-side comparison.

In some embodiments, the graphical user interface 410 may present an interactive element 814 for creating the alarm 602. For instance, interaction with the interactive element 814 may cause the graphical user interface 410 to display one more fields and/or selectable elements for receiving user input to set one or more parameters or events associated with the alarm 602. For instance, the one or more parameters may comprise a weight value range, such that the alarm 602 triggers if the weight value 110 associated with a particular animal is outside the weight value range. The one or more parameters may comprise a particular weight change occurring within a particular time period, such that the alarm 602 triggers if the animal 106 losses an amount of weight or gains an amount of weight outside of an acceptable threshold amount within the particular time (e.g., one day, one month, six months, one year, etc). The one or more parameters may comprise a daily requirement to receive the particular identifier 600, such that the alarm 602 triggers if the particular identifier 600 of the animal 106 is not received in a day (e.g., indicating that the animal 106 did not drink water or eat food during that day). The one or more parameters may comprise a location parameter, such that the alarm 602 triggers if the animal 106 is detected at a particular identification tag detector, or not detected at the particular identification tag detector, in some instances, during a particular time period. The one or more parameters may comprise a physical characteristic of a location of the one or more weight sensors 102, such as a temperature reading being outside a temperature range and/or above or below a temperature threshold, a moisture reading being outside a moisture range and/or above or below a moisture threshold, or a preset sunlight or precipitation event occurring, or other events which may impact the accuracy of the one or more weight sensors 102. Various sensors to detect the above-mentioned parameters may be installed at the one or more weight sensors 102 and/or the identification tag detector 112 to detect and report parameter input data for creating and triggering alarms 602.

In some examples, the alarm 602 may be created and applied to a particular animal profile 816 corresponding to a particular animal. For instance, the graphical user interface 410 may present one or more selectable animal profiles that, when selected 818, indicate the animal profile 426 to which the alarm 602 will be applied. In some examples, the alarm 602 may be applied to a group of one or more animals, the group being generated using one or more of the processes discussed above, or by selecting multiple of the selectable animal profiles. The alarm 602 may be applied to an entire herd of animals, such as every animal profile 426 stored in the system 100. In some instances, the graphical user interface 410 may present one or more interactive elements for determining which animal profile(s) 426 to which the alarm 602 is applied.

In some examples, alarm 602 may be set to trigger and provide a notification (e.g., a sound alert, a visual alert such as a red bubble or red number alert, a tactile alert such as a vibration, etc.) if an event or parameter of the one or more parameters occurs. In some instances, the alarm 602 triggering may cause a follow-up process to be scheduled, as discussed below in greater detail with respect to FIG. 9.

FIG. 9 illustrates an example third graphical user interface presentation 900 of the system 100. In some instances, the graphical user interface 410 may present a particular animal profile 902 (e.g., as a result of interaction with an interactive element as discussed above with respect to FIG. 8), as well as an interactive element to present information associated with the particular animal profile 902. For instance, the graphical user interface 410 may present an indicator 904 of the particular animal profile 902 being accessed (such as a particular identifier associated with the particular animal profile 902), and one or more interactive elements 906 indicating one or more time periods for which a series of weight values is to be displayed. For instance, the one or more interactive elements 906 may comprise a “today” element which, when selected, causes the graphical user interface 410 to display all weight values 110 collected for the particular animal profile 902 on the current day, a “7 day” element which, when selected, causes the graphical user interface 410 to display all weight values 110 collected for the particular animal profile 902 for the past 7 days, a “30 day” element, a “60 day” element, a “6 month” element, a “1 year” element, a “2 year” element, a “custom” element which, when selected, presents an interactive element or field to receive user input for generating a custom time period of weight values 110 to display (e.g., a range of dates or a number of days prior to the current day) associated with the particular animal profile 902, or an “all” element which, when selected causes the graphical user interface 410 to display all weight values 110 associated with the particular animal profile 902. The graphical user interface 410 may display one or more weight values 110 as a line graph, a bar chart, a list, or any other type of graphical presentation to present information in a human-readable form.

In some examples, the graphical user interface 410 may present an interactive element 908 for scheduling a follow-up process associated with a particular animal profile 426 or a group of one or more animal profiles 426. For instance, upon interaction with the interactive element 908, the graphical user interface 410 may present one or more selectable elements which correspond to creating one or more task alerts, such as a task alert to change a diet of an individual animal associated with the animal profile 426 (or a plurality of animals associated with a plurality of animal profiles), or an alert task to treat a health problem corresponding to a predetermined loss of weight over a particular time period. In some examples, the graphical user interface 410 may present an interactive element for associating the follow-up process with a triggering of a particular alarm, such that the triggering of the particular alarm automatically schedules the follow-up process and, in some instances, provides a notification that the follow-up process has been scheduled.

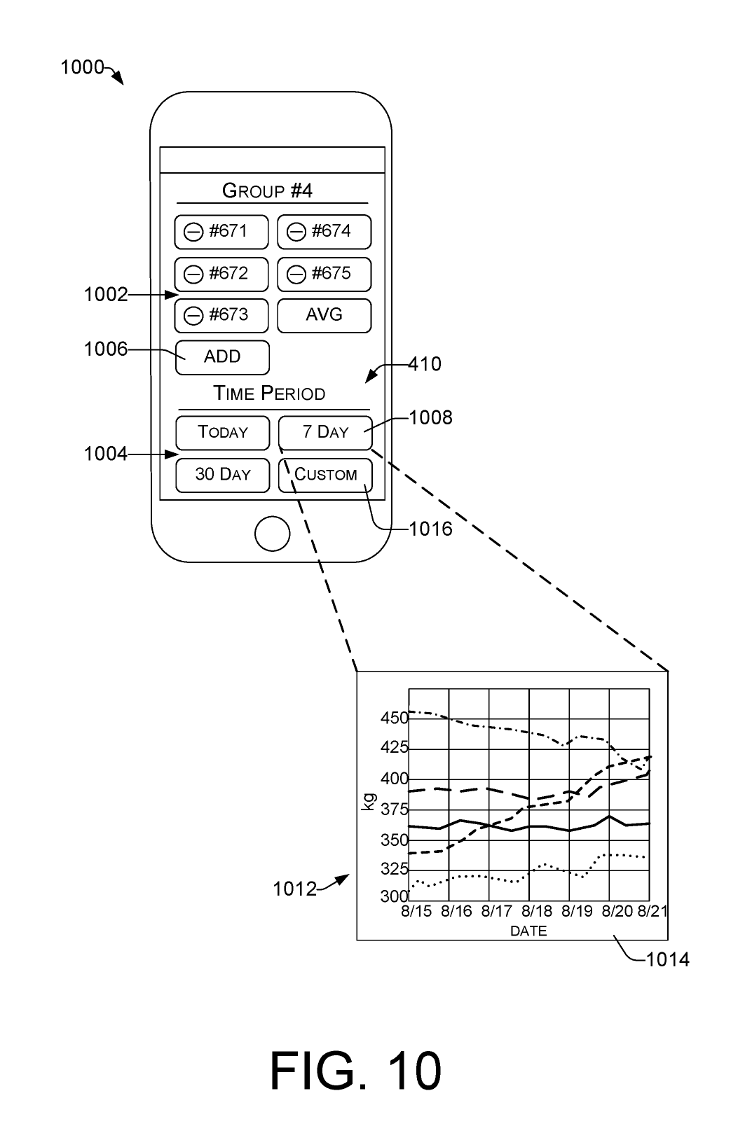

FIG. 10 illustrates an example fourth graphical user interface presentation 1000. The graphical user interface 410 may present one or more interactive elements 1002 corresponding individual animal profiles and/or identifiers of individual animals that comprise a group of animals, and one or more interactive elements 1004 for displaying weight values 110 associated with the group of animals over a particular time range. In some examples, the one or more interactive elements 1002 may comprise selectable elements corresponding to one or more animal profiles 426, such that the selectable elements may be selected to remove the one or more animal profiles 426 from the group of animals. The selectable elements may include an “add animal” element 1006 which, when selected, presents a field or selectable element for receiving user input corresponding to the animal profile 426 to be added to the group of animals.

In some instances, the graphical user interface 410 may present one or more interactive elements 1004 which, when selected, cause the graphical user interface 410 to display one or more weight values 110 associated with the group of animals that are collected over a time period corresponding to a selected time period element 1008 of the interactive elements 1004. Displayed one or more weight values 1012 may be displayed as a line graph 1014, with an x-axis corresponding to dates associated with the one or more weight values 110, and an y-axis corresponding to the one or more weight values 110. In some examples, the line graph 1014 may present an individual line corresponding to an individual animal of the group of animals. In some examples, the one or more weight values 110 may be displayed as a bar graph, as a pie chart, or as another type of infographic representation for presenting the weight values 110 over the selected period of time in a human-readable form. In some examples, the graphical user interface 410 may present an average-weight-change element which, upon selection, causes the graphical user interface 410 to present an average weight change of the group of animals, or an individual animal, over the selected time period. In some instances, the graphical user interface 410 may present an interactive element 1016 for generating a custom time period, based on user input received at an input field or selectable input feature, to correspond to the x-axis.

In some examples, the graphical user interface 410 may present an interactive element for presenting weight gain information and/or a weight loss information over a particular time period corresponding to one or more groups of animals from different suppliers, or one or more groups of animals corresponding to one or more different breeds, or based on any other information associated with the animal profiles 426 (as discussed with respect to FIG. 6). The weight gain information and/or weight loss information may be presented in the form of a bar graph or a line graph or other type of infographic representation.

Although multiple graphical user interface presentations are discussed herein as what may be interpreted as distinct presentations, any suggestion that the presentations are distinct and/or not combinable is solely for ease of understanding and brevity and is not intended to be limiting. Any and all of the graphical user interface presentations, and features of the presentations may be combined, multiplied, and modified. For instance, filters discussed with respect to FIG. 10 may be combined with displaying the group weight value presentation discussed with respect to FIG. 8. For instance, the function for creating a group of animals discussed in FIG. 8 may be combined with the one or more time periods for which a series of weight values 110 is to be displayed discussed in FIG. 9. The graphical user interface 410 may present any of the information regarding one or more animals 106 discussed above in real-time, e.g., substantially contemporaneously with the data being generated and collected. Every graphical user interface presentation, interactive element, feature, user input field, selectable item, or other element discussed herein may be combined with any other graphical user interface interactive elements, features, user input fields, selectable items, and/or other elements to, in some instances, display data of the system 100 in ways that improve animal weight monitoring, management, decision-making, supplier comparisons, breed comparisons, weight tracking, and/or scheduling follow-up processes.

### CONCLUSION

Although this disclosure uses language specific to structural features and/or methodological acts, it is to be understood that the scope of the disclosure is not necessarily limited to the specific features or acts described. Rather, the specific features and acts are disclosed as illustrative forms of implementation.

What is claimed is:

1. A system comprising:
one or more weight sensors fixed to a substantially planar platform;
an identification tag detector;
one or more processors; and
one or more non-transitory computer-readable storage media having instructions stored thereupon which are executable by the one or more processors and which, when executed, cause the system to:
receive, from the identification tag detector, an identification signal indicating that an identification tag associated with an animal is within a predetermined distance of the identification tag detector;
determine, from the identification signal, an identifier corresponding to the identification tag;
store, in the one or more non-transitory computer-readable storage media, the identifier as an identifier data file;
receive, from the one or more weight sensors, a weight signal;
transform the weight signal into a weight value;
store, in the one or more non-transitory computer-readable storage media, the weight value as a weight value data file; and
determine a weight of the animal based at least in part on associating the weight value data file with the identifier data file.

2. The system of claim 1, wherein determining the weight of the animal further comprises determining that the weight signal is received substantially contemporaneously with the identification signal.

3. The system of claim of 2, wherein the instructions, when executed, further cause the system to:
generate or receive a first timestamp associated with receiving the identification signal;
generate or receive a second timestamp associated with receiving the weight signal; and
determining the weight of the animal further comprises determining that the first timestamp is within a predetermined time range of the second timestamp.

4. The system of claim 1, wherein associating the weight value data file with the identifier data file is based at least in part on the identification tag detector detecting a tag distance associated with the identification tag, and further comprising comparing the tag distance with a predetermined distance of the identification tag detector from the one or more weight sensors.

5. The system of claim 1, wherein associating the weight value data file with the identifier data file further comprises recognizing that the identifier data file corresponds to a previously-stored identifier data file that corresponds to an animal profile data file associated with the animal.

6. The system of claim 5, further comprising identifying a previously-stored weight data file that associates an initial weight value with the animal profile data file, wherein determining the weight of the animal is further based at least in part on determining that the weight value is within a range of the initial weight value.

7. The system of claim 1, wherein the platform comprises a lattice structure to position the one or more weight sensors above a ground surface adjacent to a water trough or a feed trough.

8. The system of claim 1, wherein the identification tag comprises a passive radiofrequency identification tag.

9. A method comprising:
detecting that an identification tag is within a predetermined distance of an identification tag detector;
determining, via the identification tag detector, an identifier from the identification tag;
detecting that a load is activating one or more weight sensors and is causing the one or more weight sensors to output a weight signal;
extracting, from the weight signal, a weight value associated with the load;
analyzing, via one or more processors, the identifier and the weight value;
determining, from analyzing, that the identifier is associated with an animal; and
determining, from analyzing, whether or not the weight value is associated with the animal.

10. The method of claim 9, wherein determining whether or not the weight value is associated with the animal includes comparing a first timestamp associated with extracting the identifier to a second timestamp associated with extracting the weight value.

11. The method of claim 9, wherein the load comprises a first load and the weight signal comprises a first weight signal corresponding to a first weight value, and further comprising detecting that a second load is activating the one or more weight sensor and causing the one or more weight sensors to output a second weight signal corresponding to a second weight value; and
analyzing further comprises:
calculating, by subtracting the first weight value from the second weight value, a third weight value;
comparing the first weight value, the second weight value, or the third weight value to one or more previously-stored weight data files associated with one or more animal profile data files; and
determining that the first weight value indicates that the animal comprises a first animal activating the one or more weight sensors, and the second weight value indicates a second animal and the first animal both triggering the one or more weight sensors.

12. The method of claim 11, wherein the one or more previously-stored weight data files indicate one or more weight value ranges associated with one or more animal profile data files.

13. The method of claim 9, wherein the one or more weight sensors comprises a grid of weight sensors, and the weight signal includes information indicating a particular configuration of the grid of weight sensors that is activated.

14. The method of claim 13, wherein the particular configuration comprises a distribution of weight sensors that corresponds to stance dimensions of the animal.

15. The method of claim 9, further comprising detecting a changing weight load activating the one or more weight sensors, and determining that the changing weight load corresponds to an animal event data file that includes a weight-change profile associated with i) consumption of water; or ii) a bodily excretion.

16. The method of claim 9, further comprising receiving a request, via a graphical user interface of a mobile device, to output the weight of the animal, and, in response to receiving the request, displaying the weight of the animal.

17. An apparatus comprising:
a non-transitory computer-readable storage media having instructions stored thereupon that are executable by one or more processors and which, when executed, cause the one or more processors to:
receive, from an identification tag detector, an identifier associated with an identification tag;
receive, from one or more weight sensors, a weight signal indicating a weight value of a load activating the one or more load sensors;
associate the identifier with an animal;
associate the weight value with the animal;
present, on a display, a graphical user interface including an interactive element for requesting presentation of the weight value associated with the animal; and
present, upon an interaction with the interactive element, the weight value associated with the animal.

18. The apparatus of claim 17, wherein the instructions, when executed, further cause the processors to associate a plurality of weight values, received from the one or more weight sensors, with a plurality of identifiers, received from the identification tag detector;
wherein the interactive element comprises a first interactive element, and the graphical user interface further includes a second interactive element for requesting presentation of an average weight change over a particular period of time of a group of animals corresponding to the plurality of identifiers and the plurality of weight values.

19. The apparatus of claim 17, wherein the interactive element comprises a first interactive element, and the graphical user interface further includes a second interactive element for setting an alarm that provides an alert upon a determination that the weight value associated with the animal is outside a threshold weight range.

20. The apparatus of claim 17, wherein the interactive element comprises a first interactive element, and the graphical user interface further includes a second interactive element for requesting a presentation of a series of weight values associated with the animal over a particular period of time.
